(19) United States
(12) Patent Application Publication
(10) Pub. No.: US 2004/0248770 A1
(43) Pub. Date: Dec. 9, 2004

(54) Process for ultrafiltration of an antifungal agent

(76) Inventors: Joseph P Grau, New Brunswick, NY (US); Hsien-Hsin Tung, Edison, NJ (US)

Correspondence Address: MERCK AND CO INC, P O BOX 2000, RAHWAY, NJ, 070650907

(21) Appl. No.: 10/491,065

(22) PCT Filed: Sep. 27, 2002
(86) PCT No.: PCT/US02/30707

Related U.S. Application Data
(60) Provisional application No. 60/326,439, filed on Oct. 2, 2001.

(51) Int. Cl. A61K 38/00; G01N 33/53

(52) U.S. Cl. 514/2; 530/412; 514/254.07; 514/383; 514/397

(57) ABSTRACT

A method for the ultrafiltration of an antifungal agent using a zirconium-layered ceramic ultrafilter to reduce pyrogens is disclosed.

### BACKGROUND OF THE INVENTION

The process development of a sterile intravenous formulation of antifungal agent has required the development of a method for limiting the pyrogens and endotoxins to acceptable levels in order to meet the endotoxin specifications in the bulk material.

CANCIDAS (Caspofungin acetate) is a sterile, lyophilized product for intravenous infusion that contains a semisynthetic lipopeptide (echinocandin) compound synthesized from a fermentation product of Glarea lozoyensis. CANCIDAS is a member of a class of antifungal drugs (glucan synthesis inhibitors) that inhibits the synthesis of β (1.3)-D-glucan, an integral component of the fungal cell wall. CANCIDAS has been approved for marketing in the United States; it is indicated for the treatment of invasive aspergillosis in patients who are refractory to or intolerant of other therapies. Echinocandins of this kind are disclosed in U.S. Pat. No. 5,378,804 that issued Jan. 3, 1995; U.S. Pat. No. 5,514,650 that issued May 5, 1996; and U.S. Pat. No. 5,792,746 that issued Aug. 11, 1998. The echinocandin compounds disclosed in these patents have been prepared as described in these patents, and in patents claiming process improvements. U.S. Pat. No. 5,552,521 discloses a three-step process for preparing the compounds of the invention. U.S. Pat. No. 5,936,062 discloses an improvement of the three-step process using a boronate intermediate. Articles in the Journal of Organic Chemistry, 1999, 64, 2411-2417 and J. Med. Chem. 1994, 37, 222-225, describe an amide to nitrile dehydration of similar echinocandins using cyanuric chloride.

U.S. Pat. No. 5,104,546 discloses the use of a zirconium oxide membrane on a ceramic support with a nominal pore size of about 20 angstroms to 100 angstroms to separate pyrogens. Additionally, the use of ceramic membrane ultrafiltration to prepare water for injection has been discussed and suggested to be an economical alternative to distillation or reverse osmosis technology that has been approved by the U.S. Pharmacopeia. See “Making Water for Injection with Ceramic membrane Ultrafiltration” W. Reinholtz, Pharmaceutical Technology, 1995, 19(9) pp. 84-96.

Endotoxins, commonly known as pyrogens, are lipopolysacchardide (LPS)-protein complexes that are components of the outer membrane of Gram-negative bacteria. These molecules are high molecular weight complexes, e.q., molecular weights of about 10,000 up to 100,000 to 200,000. The use of ultra-filtration is a common technique used to filter out pyrogens. Polymer-based ultrafilters are typically used with an aqueous solution to filter the pyrogens out. Caspofungin acetate requires the use of an organic solvent for this ultrafiltration process. The ultrafiltration process was attempted using the standard polymer-based ultrafilters, which failed to work well as the filters swelled effectively plugging the membrane pores and clogging these polymer-based ultrafilters. Examples of this polymer-based ultrafilters are two ultrafilters from AG Technology and Millipore that utilize a polysulfone membrane.

The instant invention provides a method for ultrafiltration on manufacturing scale of an antifungal agent using a zirconium-layered ceramic membrane (Membralox, U.S. Filter) for pyrogen reduction via ultrafiltration in the last step synthesis of bulk drug.

### SUMMARY OF THE INVENTION

The instant invention discloses a method for ultrafiltration of an antifungal agent comprising filtering of the antifungal agent through a ceramic ultrafilter using a solvent to produce a filtered-antifungal agent with reduced endotoxin levels. An embodiment of the invention is the method for ultrafiltration of Caspofungin or its pharmaceutically acceptable salts comprising filtering of Caspofungin or its pharmaceutically acceptable salts through a zirconium-layered ceramic ultrafilter using a solvent to produce a filtered-Caspofungin or its pharmaceutically acceptable salts with reduced endotoxin levels.

### DETAILED DESCRIPTION OF THE INVENTION

A method for ultrafiltration of an antifungal agent comprising filtering of the antifungal agent through a zirconium-layered ceramic ultrafilter having a pore size of between about 0.1 μm to about 0.01 μm using a solvent to produce a filtered-antifungal agent with reduced endotoxin levels.

The method as recited above wherein the zirconium-layered ceramic filter has a pore size of between about 0.05 μm to about 0.01 μm.

The method as recited above wherein the zirconium-layered ceramic filter has a pore size of between about 0.02 μm.

The method as recited above wherein the solvent comprises water, alcohol, or a mixture of the aforementioned solvents.

The method as recited above wherein the solvent is a mixture of water and alcohol.

The method as recited above wherein the alcohol solvent is defined as C₁-C₆ alcohol.

The method as recited above wherein the alcohol solvent is selected from methanol, ethanol, n-propanol, iso-propanol, n-butanol, isobutanol, and t-butanol.

The method as recited above wherein the solvent is a mixture of water and ethanol.

The method as recited above wherein the solvent mixture of water and ethanol, is prepared using de-ionized water.

The method as recited above wherein the solvent mixture of water and ethanol, is prepared in a 92 to 8 ratio of ethanol to water.

The method as recited above wherein the solvent is water.

The method as recited above wherein the solvent is alcohol.

The method as recited above wherein the solvent is ethanol.

The method as recited above wherein the antifungal agent is a pneumocandin derivative or its pharmaceutically acceptable salts thereof.

The method as recited above wherein the antifungal agent is Caspofungin or its pharmaceutically acceptable salts thereof.

A method for ultrafiltration of Caspofungin or its pharmaceutically acceptable salts comprising filtering of Caspofungin or its pharmaceutically acceptable salts through a zirconium-layered ceramic ultrafilter having a pore size of about 0.02 μm using a solvent to produce a filtered-Caspofungin or its pharmaceutically acceptable salts with reduced endotoxin levels.

The method as recited above wherein the solvent is a mixture of water and ethanol.

The method as recited above wherein the solvent mixture of water and ethanol, is prepared in a 92 to 8 ratio of ethanol to water.

The method as recited above wherein the solvent is water.

The method as recited above wherein the solvent is ethanol.

The following examples illustrate this invention, and as such are not to be considered as limiting the invention set forth in the claims appended hereto.

### EXAMPLE 1

The ceramic filter used in the laboratory is a 0.02 μm pore size, 254 mm long zirconium layer single-lumen membrane element in a stainless steel housing (model 1T1-70). The filter and the stainless steel housing were purchased from U.S. Filter. There are two outlets for both the retentate and permeate sides of the filter system. During the experiments, for both the retentate and permeate sides of the filter system, one port is plugged and the other port is equipped with a Luerlok fitting.

Initially, the filter system is depyorgenated by soaking with 0.5N NaOH solution over 30 minutes, followed by a dilute NaOCl solution at a concentration of 4 mg/ml. After draining off the NaOCl solution, the ultrafiltered depyrogenated water is injected from the retentate side until the pH of permeate becomes 7 to 8 in order to flush out the remaining NaOCl in the system. The filter system is then placed in an oven at 250° C. for 16 hrs.

The feed solution is prepared by injecting 4 ml of ultrafiltered depyrogenated water into a vial containing 10,000 EU/mL USP endotoxin (US Pharmacopeia, Lot G). The vial is then vigorously shaken for 30 minutes using a vortex shaker to ensure proper dispersion of endotoxin. The solution is poured into a 50 ml polystyrene centrifuge tube containing about 41 ml ethanol. To minimize the residual liquid retained in the vial, an additional 5 ml of ethanol is added to the vial, vigorously mixed with the vortex mixer, and poured into the same 50 ml polystyrene centrifuge tube.

The polystyrene tube is vigorously mixed for 30 minutes using the vortex mixer. This yields a feed solution with 92/8 ethanol/water composition and an endotoxin level of about 200 endotoxin unit (EU) per ml of feed solution.

To confirm the endotoxin level in the unfiltered feed solution, 5 ml of the feed solution is poured into a polystyrene tube and is blown gently to dryness with HEPA-filtered nitrogen at room temperature to remove all solvents, especially the alcohol. The sample is reconstituted with ultrafiltered depyrogenated water and L.A.L. (Limulus Amebocyte Lysate) assay is conducted.

The LAL assays were conducted in accordance with 1987 FDA Guidelines and the Associate of Cape Cod operations manual. The tests were conducted utilizing turbidimetric techniques with the LAL 5000 and the Pyros™ for Windows software, version 1.01. A series of dilution of the sample up to 160-X is performed until there was no significant interference observed at this dilution, and spike recoveries remained in the acceptable range of 50-105% of the nominal concentration. The limit of detection of the instrumentation is 0.001 EU/mL (or equivalently 0.04 to 0.08 EU/mg of Caspofungin acetate depending upon the noise level).

To filter the feed solution, the remainder of the 45 ml unfiltered feed solution is injected into the retentate side of the filter with a sterile polystyrene syringe. Permeate is collected in polystyrene tubes. Similar to the above procedure, 5 ml of the collected permeate is blown to dryness and L.A.L. assay is conducted as unfiltered sample.

Table 1 below shows the results of LAL assay before and after the ultra-filtration. The data demonstrated a significant 3 log₁₀ endotoxin reduction from 128 EU/mL to 0.127 EU/mL in the permeate.

TABLE 1
L.A.L. Results (before and after filtration)

| Sample # | Description | L.A.L assays EU/mL |
|---|---|---|
| 1 | Unfiltered feed | 194 |
| 2 | Filtrate | Less than 7.81 |
| 3 | Unfiltered feed | 128 |
| 4 | Filtrate | 0.127 |

### EXAMPLE 2

A process stream sample containing Caspofungin acetate, versus synthetic feed sample above, was taken from a large-scale batch and filtered in the laboratory. Table 2 shows the result before and after the filtration.

| | | L.A.L assays, EU/mg of |
|---|---|---|
| Sample # | Description | Caspofungin acetate |
| 1 | Unfiltered process stream sample | 0.33 |
| 2 | Filtered | <0.04 |
| | | (below detection limit) |

### EXAMPLE 3

A factory-size ceramic filter (model 1R19-40) from U.S. Filter was used in large-scale campaign. The ceramic filter has the same 0.02 μm pore size, with 1020 mm long zirconium layer 19-lumen membrane element in a stainless steel housing. To depyrogenate the system, the ceramic filter system is flushed with NaOH solution, NaOCl solution and ultrafiltered depyprognated water, similarly to the laboratory procedure as described in Example 1. Following the flush of ultrafiltered depyrogenated water, the filter system is flushed with a solvent mixture of ethanol/water of about 92/8 composition.

The performance of the factory-size ceramic filter was 100% successful. L.A.L. assays of all final pure Caspofungin acetate (nine batches, 50-60 liter of solution/batch) were <0.04 to <0.08 EU/mg of Caspofungin acetate, which is below the detection limit of L.A.L. assays. The campaign included one batch, which was conducted using only de-ionized water, instead of ultrafiltered depyrogenated water, to challenge the ceramic filtration system with microbial burden. Unfiltered dyprogenated water for this period were measured at 1350 cfu/ml to 5150 cfu/ml (colony forming unit/ml of water).

What is claimed is:

1. A method for ultrafiltration of an antifungal agent comprising filtering of the antifungal agent through a zirconium-layered ceramic ultrafilter having a pore size of between about 0.1 μm to about 0.01 μm using a solvent to produce a filtered-antifungal agent with reduced endotoxin levels.

2. The method as recited in claim 1, wherein the zirconium-layered ceramic filter has a pore size of between about 0.05 μm to about 0.01 μm.

3. The method as recited in claim 2, wherein the zirconium-layered ceramic filter has a pore size of between about 0.02 μm.

4. The method as recited in claim 3, wherein the solvent comprises water, alcohol or a mixture of the aforementioned solvents.

5. The method as recited in claim 4, wherein the solvent is a mixture of water and alcohol.

6. The method as recited in claim 5, wherein the alcohol solvent is defined as C₁-C₆ alcohol.

7. The method as recited in claim 6, wherein the alcohol solvent is selected from methanol, ethanol, n-propanol, iso-propanol, n-butanol, isobutanol, and t-butanol.

8. The method as recited in claim 7, wherein the solvent is a mixture of water and ethanol.

9. The method as recited in claim 8, wherein the solvent mixture of water and ethanol, is prepared using de-ionized water.

10. The method as recited in claim 9, wherein the solvent mixture of water and ethanol, is prepared in a 92 to 8 ratio of ethanol to water.

11. The method as recited in claim 4, wherein the solvent is water.

12. The method as recited in claim 4, wherein the solvent is alcohol.

13. The method as recited in claim 14, wherein the solvent is ethanol.

14. The method as recited in claim 1, wherein the antifungal agent is a pneumocandin derivative or its pharmaceutically acceptable salts.

15. The method as recited in claim 14, wherein the antifungal agent is Caspofungin or its pharmaceutically acceptable salts thereof.

16. A method for ultrafiltration of Caspofungin or its pharmaceutically acceptable salts comprising filtering of Caspofungin or its pharmaceutically acceptable salts through a zirconium-layered ceramic ultrafilter having a pore size of about 0.02 μm using a solvent to produce a filtered-Caspofungin or its pharmaceutically acceptable salts with reduced endotoxin levels.

17. The method as recited in claim 16, wherein the solvent is a mixture of water and ethanol.

18. The method as recited in claim 17, wherein the solvent mixture of water and ethanol, is prepared in a 92 to 8 ratio of ethanol to water.

19. The method as recited in claim 18, wherein the solvent is water.

20. The method as recited in claim 19, wherein the solvent is ethanol.
